(19) United States
(12) Patent Application Publication
(10) Pub. No.: US 2024/0363231 A1
(43) Pub. Date: Oct. 31, 2024

(54) SYSTEMS, PLATFORMS, AND METHODS FOR REDUCING NETWORK BANDWIDTH ASSOCIATED WITH CONNECTING PATIENTS AND MEDICAL PROVIDERS

(71) Applicant: Abdullaibrahim Abdulwaheed, Holbrook, MA (US)

(72) Inventor: Abdullaibrahim Abdulwaheed, Holbrook, MA (US)

(21) Appl. No.: 18/306,302

(22) Filed: Apr. 25, 2023

(51) Int. Cl. G16H 40/20; G16H 80/00

(52) U.S. Cl. CPC G16H 40/20; G16H 80/00

(57) ABSTRACT

A system and method are provided to reduce network bandwidth related to determining medical providers. The method includes receiving one or more patient criteria and one or more patient human factors associated with healthcare from a patient. A query to a database is transmitted where the query includes the patient criteria and the human factors. A list of medical providers is received from the database based on the query and the list of medical providers is transmitted to the patient. In some embodiments, the patient human factors and the patient criteria can be supplemented by a machine learning server. In some embodiments, the patient human factors and the patient criteria can be generated by an analytical server processing audio and/or visual data collected from the patient.

### BACKGROUND OF THE INVENTION

### Field of the Invention

The present invention relates broadly to computer networks, and more particularly, the present invention relates to reducing network bandwidth of a computer network by facilitating matching between medical providers and patients over the computer network.

### Current State of the Art

When searching for a medical provider (e.g., a doctor, a surgeon, a dentist, a therapist), patients traditionally use two criteria to search for their medical provider. The first is that the provider is in their insurance network and the second is the distance the patient is located from the medical provider.

Often, a patient calls the medical provider to ask questions prior to making an appointment to see if the medical provider is a good fit for them. Or, in some cases, the patient creates an appointment with a medical provider only to later find out that the provider is not a good fit and then resumes searching for a new provider. Equally, providers may find patients being of a poor fit. The providers perspective in patient selection is often overlooked. Every time a patient contacts or searches for a medical provider, or preforms a new search, the patient is using increasingly more bandwidth by making calls over a computer network that uses voice over internet protocol (VOIP) or transmitting queries over the internet which creates network congestion and uses additional network bandwidth. As this is done millions of times each day, the load on networks related to searching and contacting medical providers is significant. It would therefore be desirable to provide a system to allow patients to determine if a medical provider is a good fit, and for a provider to better determine fit, while reducing network usage.

### BRIEF SUMMARY

The possible embodiments described herein generally relate to systems and methods that facilitate matching between medical providers and patients over a computer network for the purpose of reducing network bandwidth of the computer network.

According to some embodiments, the disclosed systems include a patient device having an application installed and a provider device having an application installed. The application can take the form of one or more computer programs or software instructions.

According to some embodiments, the patient device and the provider device can be computing devices, including but not limited to, smartphones, tablets, laptop computers, and desktop computers.

According to some embodiments, the system further includes a central server having a processor, a memory, and one or more programs stored within memory for reducing network bandwidth associated with connecting patients and medical providers by facilitating matching between medical providers and patients over a computer network. The central server can be communicatively coupled to the patient device via the installed application and to the provider device via the installed application.

According to some embodiments, when the one or more programs stored within the central server's memory are run on the processor, the central server is configured to receive information from the patient device and the provider device. The received information from the patient device and the provider device can be stored in the server's memory.

According to some embodiments, the central server is a web server.

According to some embodiments, the central server is a cloud server.

According to some embodiments, the patient device and provider device have a global positioning system (GPS) tracking system.

According to some embodiments, the central server is communicatively connected to an analytical server and a machine learning server over a network.

According to some embodiments, the central server, the analytical server, and the machine learning server are the same device.

### BRIEF DESCRIPTION OF THE DRAWINGS

The accompanying drawings, which are included to provide a further understanding of the invention and are incorporated in and constitute a part of this specification, illustrate embodiments of the invention and, together with the description, serve to explain the principles of the invention.

FIG. 1 is a diagram illustrating the system for reducing network bandwidth associated with connecting patients and medical providers according to a first embodiment of the invention.

FIG. 2 is a diagram of example components of a computing device utilized by the system of FIG. 1.

FIG. 3 illustrates an exemplary patient interface utilized by the system of FIG. 1.

FIG. 4 is a flowchart illustrating the steps of a method for reducing network bandwidth of a computer network according to a first embodiment of the invention.

FIG. 5 is a flowchart illustrating the steps of a method for reducing network bandwidth of a computer network according to a second embodiment of the invention.

FIG. 6 illustrates two exemplary tables according to an embodiment of the invention.

### DETAILED DESCRIPTION

In the following description, to better understand the aforementioned purposes, features, and advantages of the present invention, numerous specific details are set forth in order to provide a thorough understanding of the claimed subject matter. It should be noted that these details and examples are provided to merely aid in understanding the descriptions, and they do not, in any way, limit the scope of the present invention. The present invention can also be implemented in other modes different from those described herein and the present invention is not limited to the specific embodiments disclosed below.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The specification may refer to “an”, “one” or “some” embodiment(s) in several locations. This does not necessarily imply that each such reference is to the same embodiment(s), or that the feature only applies to a single embodiment. A single feature of different embodiments may also be combined to provide other embodiments.

Furthermore, as used herein, the singular forms “a”, an” and “the” are intended to include the plural forms as well, unless expressly stated otherwise, It will be further understood that the terms “includes”, comprises “, including” and/or “comprising” when used in this specification, specify the presence of stated features, integer steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

The computer-readable storage medium (“memory”) can be a tangible device that can retain and store instructions for use by an instruction execution device. The computer-readable storage medium may be, without limitation, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including, but not limited to, an object-oriented programming language such as Python, Java, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer-readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus, or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Unless otherwise defined, all terms (including technical and scientific terms) used herein have the same meaning as commonly understood by one of ordinary skill in the art to which this disclosure pertains. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The present embodiments relate to a system and method associated with minimizing searches for medical providers to reduce computer usage and to save on network bandwidth. The present embodiments may further relate to a system where both patients and medical providers provide information to facilitate finding a provider that is a good fit for both the medial provider and the patient as well as to minimize search time to reduce bandwidth and processor usage. In other words, the present system may help a doctor find a best fitting patient and help the patient find a best fitting doctor for the patient all while reducing network bandwidth and processor usage.

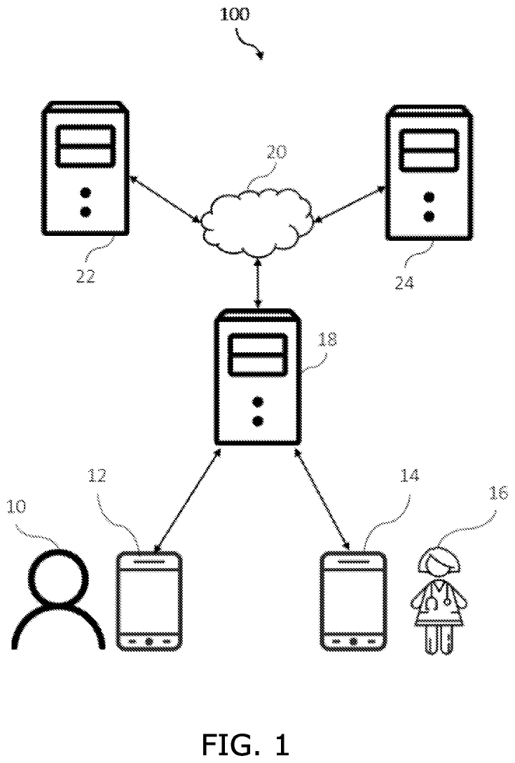

FIG. 1 is a diagram illustrating the system for reducing network bandwidth associated with connecting patients and medical providers according to a first embodiment of the invention. Patients 10 use patient devices 12 to interact with a central server 18 that is communicatively coupled with an analytical server 24 and a machine learning server 22 using a network 20. Similarly, providers 16 use provider devices 14 to interact with the central server 18.

The patient device 12 and the provider device 14 can be computing devices, including but not limited to, smartphones, tablets, laptop computers, and desktop computers. The patient device 12 and the provider device 14 are configured with a user-facing interface, a means of user data entry, and a means of recording user audio data and/or visual data.

According to some embodiments, the patient devices have an application installed and the provider devices have an application installed. The application can take the form of one or more computer programs or software instructions.

The system further includes a central server 18 having a processor, a memory, and one or more programs stored within memory for reducing network bandwidth associated with connecting patients and medical providers by facilitating matching between medical providers and patients over a computer network. In some embodiments, the central server 18 can be communicatively coupled to the patient device 12 via the installed application and to the provider device via the installed application.

When the one or more programs stored within the central server's memory are run on the processor, the central server is configured to send and receive information to and from patient devices 12 and provider devices 14. The received information from patient devices 12 and provider devices 14 can be stored in the central server's memory. Likewise, the received information from the central server 18 can be stored in the memory of patient devices 12 and in the memory of provider devices 14.

According to some embodiments, the central server 18 is a web server.

According to some embodiments, the central server 18 is a cloud server.

According to some embodiments, patient devices 12 and provider devices 14 each have a global positioning system (GPS) tracking system.

According to some embodiments, the central server 18 is communicatively connected to an analytical server 22 and a machine learning server 24 over a network 20.

According to some embodiments, the central server 18, the analytical server 22, and the machine learning server 24 are the same device.

According to the first embodiment of the invention, all data collected from patients 10 via patient devices 12 are combined into a consolidated patient database on the central server 18. Likewise, all data collected from providers 16 via provider devices 14 are combined into a consolidated provider database on the central server 18. In some embodiments, the central server may operate in a networked environment using logical connections to one or more remote computers. The remote computer (or computers) may be another personal computer, a server, a router, a network PC, a peer device, or other common network node including, but not limited to, the aforementioned patient devices 12 and provider devices 14.

The consolidated patient database may include, without limitation, data values for patient criteria such as patients' insurance coverage, spoken language(s), and maximum distance that the patient would be willing to travel to reach a provider, patient human factors such as patients' religion, medical philosophy, cultural background, expectations, age range, and desired size of provider. In some embodiments, data values may further be labeled as critical or non-critical in the consolidated patient database.

In some embodiments, a consolidated patient database is not utilized by the system. Rather, data values collected from patients 18 are stored temporarily within the various memories of the system and used only to the extent necessary to perform the disclosed methods.

The consolidated provider database may include, without limitation, data values for provider criteria such as providers' number of malpractice suits, number of publications, specialty, ranking, availability, medical philosophy, types of accepted insurance, practice style, size, cultural background, and a personal profile including the provider's gender and age.

The consolidated patient database and the consolidated provider database are coupled to the analytical server 22 and can be updated in real-time. Configurable instructions for analysis of the data (partially or fully incorporated in the analytical server 22) having differentials calculations logic programmed therein can receive as input patients' data, providers' data from the central server 18.

In some embodiments, the analytical sever 22 further includes configurable instructions for analysis of audio and/or visual data generated by a patient 10 using a patient device 12 wherein the audio and/or visual data is transcribed to create a keyword database to be stored on the central server 18. Likewise, in some embodiments, the analytical sever 22 further includes configurable instructions for analysis of audio and/or visual data generated by a provider 16 using a provider device 14 wherein the audio and/or visual data is transcribed to create a keyword database to be stored on the central server 18. Keywords contained in a keyword database associated with a patient or provider are used by methods described herein.

In some embodiments, the analytical sever 22 further includes configurable instructions for analysis of patient provided websites and social media posts or reviews to create a keyword database to be stored on the central server 18. Likewise, in some embodiments, the analytical sever 22 further includes configurable instructions for analysis of provider provided websites and social media posts or reviews to create a keyword database to be stored on the central server 18.

The invention, preferably, incorporates artificial intelligence, fuzzy logic, machine learning and the like for improving potential patient-medical provider matches. The consolidated patient database and the consolidated provider database are coupled to the machine learning server 24 and can be updated in real-time. The data stored in the consolidated patient database, the data stored in the consolidated provider database, and the data generated by the analytical server 22 is used by the machine learning server 24 to create machine learning techniques. Configurable instructions for applying the machine learning techniques to the data (partially or fully incorporated in the machine learning server 24) having differentials calculations logic programmed therein can receive the aforementioned data from the central server 18 and the analytical server 22. Accordingly, the machine learning server 24 can apply the developed machine learning techniques to all data stored in the aforementioned databases and data sources.

In some embodiments, the developed machine learning techniques can generate additional keywords and/or refine the criteria for matching patients and providers. For example, a patient's response to whether the patient has an automobile or other ready transportation may limit provider matches to those within easy access to public transportation or prompt whether/how to limit the geographic area for a provider match. To this end, a pre-trained, neural language model, such as BERT, T5 or other state of the art architectures, may capture semantic similarities. This will provide a less labor-intensive process, which has also achieved parity with humans on semantic matching between key terms and phrases.

According to some embodiments, the central server, the analytical server, and the machine learning server are the same device.

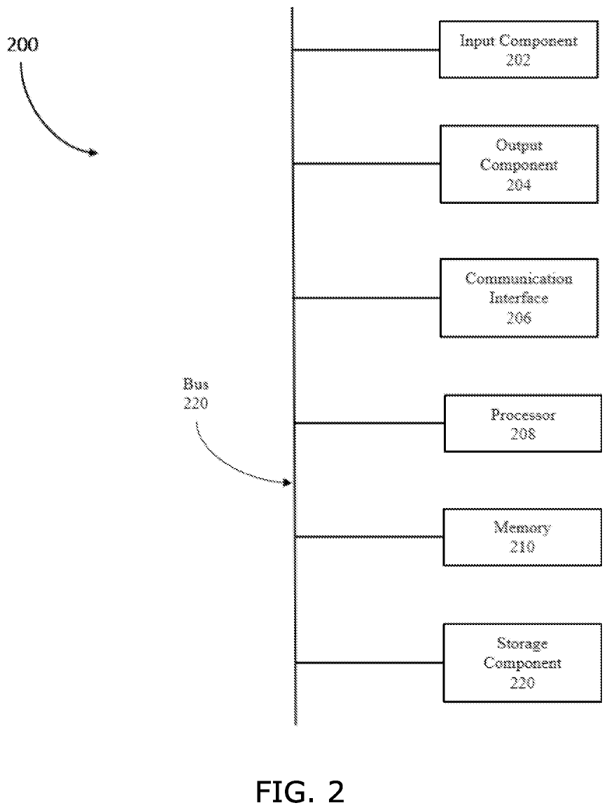

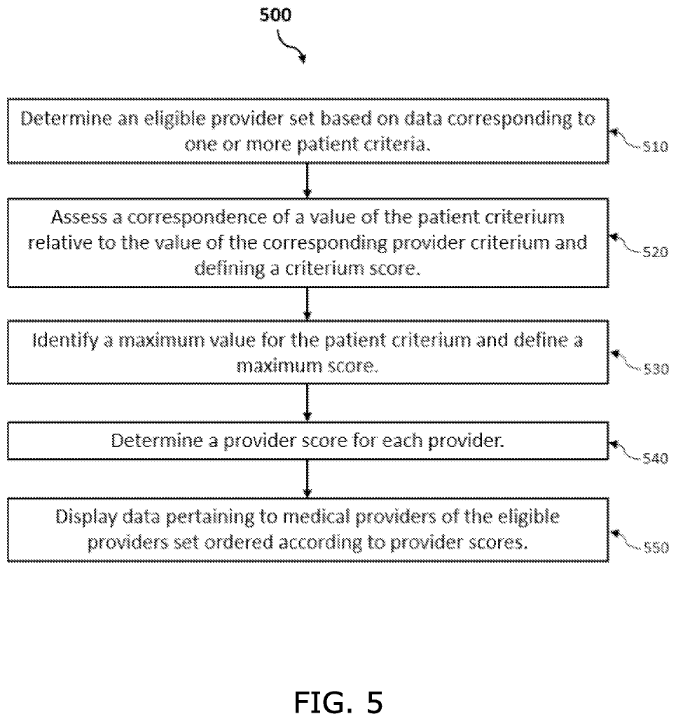

FIG. 2 is a diagram of example components of a computing device 200 utilized by the system of FIG. 1. Computing device 200 may correspond to the first user device 12, the second user device 14, the central server 18, the analytical server 22, and the machine learning server 24. In some implementations, the aforementioned devices and servers may include one or more devices 200 and/or one or more components of device 200. As shown in FIG. 5, device 200 may include an input component 202, an output component 204, a communication interface 206, a processor 208, a memory 210, a storage component 212, and a bus 214.

The bus 214 includes a component that permits communication among the components of device 200. Processor 208 is implemented in hardware, firmware, or a combination of hardware and software. Processor 208 is a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 208 includes one or more processors capable of being programmed to perform a function. Memory 210 includes a random access memory (RAM), a read only memory (ROM), and/or another type of dynamic or static storage device (e.g., a flash memory, a magnetic memory, and/or an optical memory) that stores information and/or instructions for use by processor 208.

Storage component 212 stores information and/or software related to the operation and use of device 200. For example, storage component 212 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive.

Input component 202 includes a component that permits device 200 to receive information, such as via user input (e.g., a touch screen display, a keyboard, a keypad, a mouse, a button, a switch, and/or a microphone). Additionally, or alternatively, input component 202 may include a sensor for sensing information (e.g., a global positioning system (GPS) component, an accelerometer, a gyroscope, and/or an actuator). Output component 204 includes a component that provides output information from device 200 (e.g., a display, a speaker, and/or one or more light-emitting diodes (LEDs)).

Communication interface 206 includes a transceiver-like component (e.g., a transceiver and/or a separate receiver and transmitter) that enables device 200 to communicate with other devices, such as via a wired connection, a wireless connection, or a combination of wired and wireless connections. Communication interface 206 may permit device 200 to receive information from another device and/or provide information to another device. For example, communication interface 206 may include an Ethernet interface, an optical interface, a coaxial interface, an infrared interface, a radio frequency (RF) interface, a universal serial bus (USB) interface, a wireless local area network interface, a cellular network interface, and/or the like.

Device 200 may perform one or more processes described herein. Device 200 may perform these processes based on processor 208 executing software instructions stored by a non-transitory computer-readable medium, such as memory 210 and/or storage component 212. A computer-readable medium is defined herein as a non-transitory memory device. A memory device includes memory space within a single physical storage device or memory space spread across multiple physical storage devices.

Software instructions may be read into memory 210 and/or storage component 212 from another computer-readable medium or from another device via communication interface 206. When executed, software instructions stored in memory 210 and/or storage component 212 may cause processor 208 to perform one or more processes described herein. Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 2 are provided as an example. In practice, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

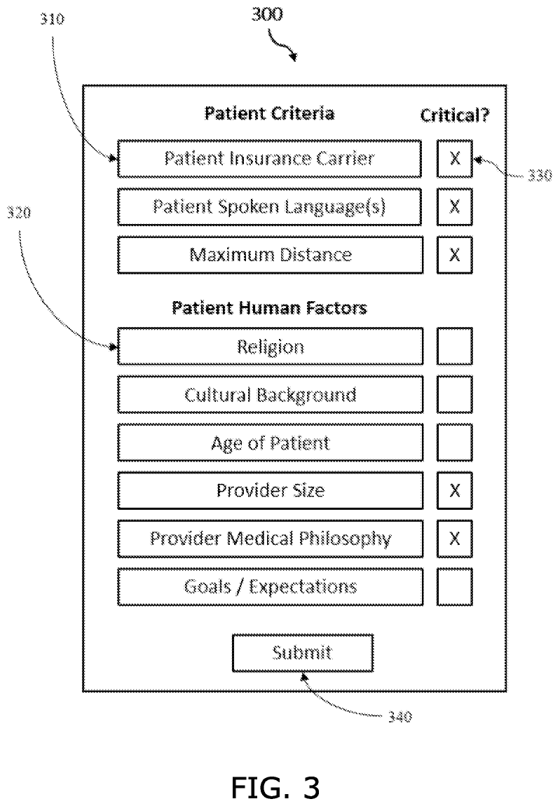

FIG. 3 illustrates an exemplary patient interface 300 utilized by the system of FIG. 1. The patient interface 300 may include a plurality of patient criteria 310, a plurality of patient human factors 320, selectable critical designators 330 for the criteria and factors, and a submit button 340. Patients can access the interface 300 using patient devices via the communication interface 206 of a patient device. The input component 202 may be used by a patient to input their information to be received by the device 200.

For illustrative purposes the plurality of patient criteria 310 may include, but are not limited to, at least one of a patient insurance carrier, a location of a patient, and a distance that a patient will be willing to travel to reach a medical provider. For example, some patients may be required to use a particular insurance carrier (e.g., Aetna, Cigna, Anthem) that is part of a benefit package supplied to the patient by an employer. Thus, when utilizing the present system, the patient would select their particular insurance carrier on the patient interface 300.

The population density of a local area, the location of the patient, and the distance that a patient is willing to travel may be criteria used to determine a location of a preferred medical provider. For example, if the patient indicates that they are located in Framingham, Massachusetts and are willing to travel 10 miles, this criterion will be used to find a provider within the selected distance of the selected location. The patient spoken language(s) may include one or more languages that the patient is comfortable speaking in. For example, some patients are more comfortable speaking in a language other than English. Patients may want to ensure that they understand the medical advice and instructions from a medical provider. Accordingly, patients prefer medical providers that can communicate with them in their preferred language.

In some embodiments, particular criteria may be indicated as critical by selecting the corresponding critical designator 330 on the patient interface 300. If a particular criterion is not met, then the medical provider should not be listed in the results of the database query. For example, a critical criterion may include state licensure, a gender of the provider, a language spoken by the provider and/or an age range of the provider. In some embodiments, multiple “critical” criteria may be indicated in the database query.

The patient human factors 320 relate to the factors that are used to refine the matching between the patient and the medical provider that go beyond the patent criteria 310. The patient human factors 320 may be used to quantify and optimize the collaborative process between patients and medical providers for an in office (on site) or online real-time medical appointment.

The patient human factors 320 vary widely according to a patient's region, culture, needs, wants and medical issues. In some embodiments, the patient human factors may include one or more of patient expectations, a patient philosophy associated with treatments, patient personal information, patient availability and provider ability, provider philosophy and practice style, prover practice size, culture, provider personal profile and location. For example, patient expectations might include, but are not limited to, an expected outcome from a visit with a medical provider (e.g., pharmaceuticals, homeopathic solutions, surgical remedies). A patient philosophy may include, but is not limited to, care philosophies such as how care for a patient is achieved such as recuperation at home vs. recuperation in a medical facility, self-help vs. more medical intervention, physical exercise vs. medications, etc. In some embodiments, human factors may relate to if a patient prefers that the medial provider makes all medical decisions, that the patient makes all medical decisions, or that the patient prefers collaborative decision-making process with the medical provider.

In some embodiments of the present system, the patient human factors 320 may further include information associated with a medical provider wherein the information may include insurance information, practice size, location information and specialization information. The information may further include publications, such as papers related to a specialty and/or relevant to a patient's case. The information may further include information about the number of malpractice suits that involved the provider within a range of time. The information may further include provider rankings and affiliated research institutions. The keywords in the database may be transcribed from an audio or video file where a medical provider answer questions related to medical philosophy and this audio and/or video file may be automatically transcribed into a database file of keywords and/or phrases associated with the medical provider. The first database may also include provider criteria such as, but not limited to, provider insurance carrier information, a provider location, and a provider spoken language. Provider human factors may include, but are not limited to, one or more of a provider philosophy, a provider practice style, a provider size, a provider cultural background, and a provider personal profile such as age, height, weight, religion, and religious beliefs.

Once a patient has properly provided the required information using the patient interface 300, said paid may click on/select the submit button 340 to send the various data values from their patient device to the central server and/or analytics server and/or machine learning server for storage and/or processing.

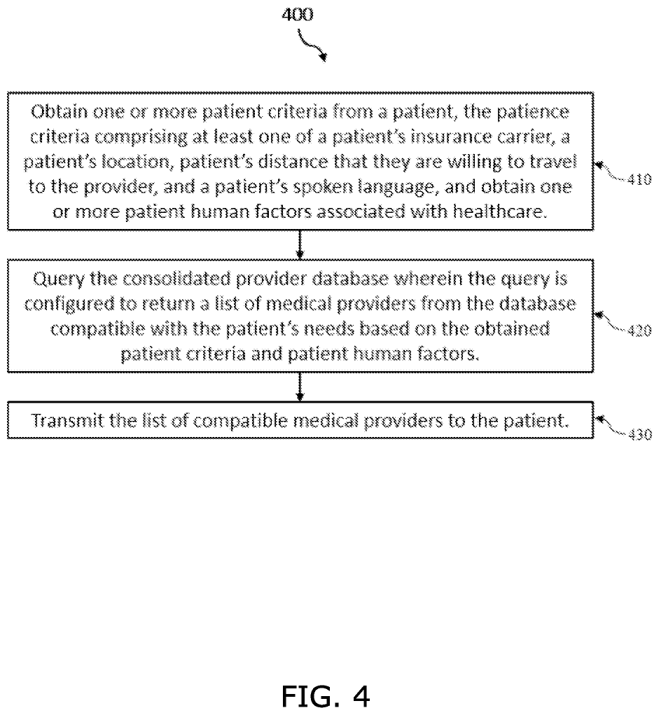

FIG. 4 is a flowchart illustrating the steps of the method 400 for reducing network bandwidth of a computer network according to a first embodiment of the invention. The system of the present invention for reducing network bandwidth of a computer network includes a server or group of servers (a central server, an analytics server, and a machine learning server), on which operations are performed according to the method, comprising the following steps.

In the preferred embodiment, the consolidated provider database stored on the central server is populated by a plurality of values associated with a plurality of medical providers input by the plurality of medical providers using their provider devices. The consolidated provider database contains a complete provider set that represents all available providers using the provided system that a patient can match with.

In step 410, a patient's data values for a plurality of patient criteria and a plurality of patient human factors are obtained from a patient inputting their data using the patient interface on their patient device and submitting the input data values. This process may be performed by the central server in conjunction with the patient device (e.g., running an application or running a software program provided by the central server.)

In some embodiments, patient data values may be obtained/extracted by the analytical server from a transcription of patient input audio and/or visual data wherein the input audio and/or visual data is obtained using the patient device.

Next, in step 420, the consolidated provider database is queried wherein the query is configured to return a list of medical providers from the database compatible with the patient's needs based on the obtained patient criteria and obtained human factors from the patient.

Finally, in step 430, the list of compatible medical providers is transmitted from the central server to the patient device and displayed to the patient using the patient interface.

FIG. 5 is a flowchart illustrating the steps of the method 500 for reducing network bandwidth of a computer network according to a second embodiment of the invention. The system of the present invention for reducing network bandwidth of a computer network includes a server or group of servers (a central server, an analytics server, and a machine learning server), on which operations are performed according to the method, comprising the following steps.

The central server of the system further includes a keyword database associated with a given patient or a given provider. In some embodiments, keywords are obtained/extracted by the analytical server from a transcription of patient or provider input audio and/or visual data wherein the input audio and/or visual data is obtained using the patient device. In some embodiments, additional keywords can be generated by the machine learning server using developed machine learning techniques.

In step 510, an eligible provider set is determined from the complete provider based on determining a score associated with criteria and keywords received from the patient and then matched to a provider's information in a provider database. In some embodiments, scoring is calculated by adding one point when the patient's answers and the provider's answers align. In some embodiments points are added based on a minimum distance from the provider to the patient. In some embodiments distance points may be created based on ranges such as, under 5 miles, 5-10 miles, 10-20 miles, over 20 miles between a patient and a provider. In some embodiments points are added based on a key word match between a human factor identified by the patient and keywords associated with the provider in the provider database. In some embodiments points are added based on a keyword match between a human factor identified by the patient and online reviews or online posts associated with the provider. For such embodiments, keyword matching is used for criteria such as philosophy where entries may be free form or created via speech to text.

In some embodiments, patients and providers may each designate some factors as critical wherein providers and patients must be in sync on critical factors in order to attain any matching points. For example, if a patient identifies language as a critical factor, then only providers with the required language skill maybe eligible for any matching points. Similarly, a provider may identify a factor as critical. For example, a provider may only be willing to see patients that speak English. As such, the provider will not be matched with a patient that does identify as English speaking.

The matching score may range from zero to no set maximum. This is because there are variabilities in the number of questions answered by both providers and patients. In addition, transcription of patient or provider audio and or/visual data introduces the potential for additional scoring opportunities based on keywords.

The matching score may be presented as a percentage of matched answers out of a total number of questions. In some embodiments, this matching percentage may be calculated based on the following formula:

Matching Percentage=(matching points associated with an individual provider/highest points achieved based on all providers returned in the database query)×100. For example, if the highest patient match between a patient and a provider is 15 points, that provider will be given a 100 score. If a second provider matches only 10 points with a patient, that provider will be given a 66.6 score. As such, this matching percentage identifies an optimal score for each provider that are is at a specific point in time.

### Example

Features, n total: [Location, Language, Profession Trait, . . . , [Key Phrases/Words], Day Available, Philosophy]
Feature Values: (Initially defined by domain experts): [β1, β2, β3, . . . , [βn-2], βn-1, βn
Patient Features: [MA, English*, Empathetic, [′Loves Kids′, ‘Listens’], Saturday, Collaborative]. Note the critical preference of matching on language denoted by *.

### Ranking Doctors

The function 1 (Doctor's language is English) represents a value of 1 if the Doctor's language is English, 0 otherwise.
Doctor A Features: [MA, English, Stern, . . . , [‘children’, ‘attentive’], Sunday, Paternal]
Similarity Overlap: [1, 1, 0, . . . , [0.6, 0.99], 0, 0]. The decimal values represent the ‘semantic similarity’ between the patient's and doctor's key words/phrases.
CCM Score: Sum ([1 β1, 1 β2, 0 β3, . . . , [0.6 βn-2, 0.99 βn-2], 0 βn-1, 0 βn])*1 (Doctor's language is English)
CCM Ratio: Above Score/Sum ([β1, β2, β3, . . . , [βn-2, βn-2], βn-1, βn])

The ratio represents Doctor A's score over the summation of all the weights, which assumes a hypothetical ideal match.
Doctor B Features: [MA, Spanish, Empathetic, [‘dislikes kids’, ‘listens’], Saturday, Collaborative]
Similarity Overlap: [1, 0, 1, . . . , [−1, 0.99], 1, 1]. The decimal values represent the ‘semantic similarity’ between the patient's and doctor's key words/phrases.
CCM Score: Sum ([1β0β1, 0β2, β3, . . . , [−1βn-2, 0.99 βn-2], 1βn-1, 1βn])*1 (Doctor's language is English). This values equals 0 as the doctor's language is not 0, so the sum is multiplied by 0.
CCM Ratio: Above Score/Sum ([β1, β2, β3, . . . , [βn-2, βn-2], βn-1, βn])=0

The ratio represents Doctor B's score over the summation of all the weights, which assumes a hypothetical ideal match.

Processing logic for the foregoing analyses may be represented as follows:

### Algorithm 1 ML Initialization Stage

1: Input: fβ representing CCM function with β set by domain experts as discussed in preceding section, dataset x∈D with x=[x1, . . . , xn] representing n features defined above, neural model fθ, loss function L
2: while fθ not converged do
3: x˜D
4: y←fβ(x)
5: ŷ←fθ(x)
6: loss←L (y, ŷ)
7: θ←θ−α∇L (y, ŷ)
8: end while
   Algorithm 2 Updating Model with User Feedback
1: Input: dataset x, y∈D with x=[x1, . . . , xn] representing n features defined above and y as user feedback, neural model fθ, multitask head fω, loss functions Lθ, Lω, stopping criteria S
2: while not S do
3: (x, y)i, (x, y)j˜D
4: Train model to learn relevance priority
5: ŷθi, ŷθj←fθ(xi), fθ(xj)
6: Train model on multitask like/dislike prediction
7: for x∈{xi, xj} do
8: ŷω←fω(x)
9: end for
10: Update weights θ, ω
11: θ←θ−α∇(Lθ(yi, yj, ŷi, ŷj)+Lω(yω, y))
12: ω←ω−α∇Lω(yω, y)
13: end while

Next, steps 520, 530, and 540 can be performed by the following algorithm:

For each patient in the eligible provider set:

For each provider criterion that corresponds with a patient criterion:

Assessing a correspondence of a value of the patient criterion relative to the value of the corresponding provider criterion and defining a criterion score, identifying a maximum value for the patient criterion and defining a maximum score, dividing a sum of the criteria scores by a sum of the maximum scores that defines a provider score for that provider.

Finally, at step 550, the patient device 12 receives the list of eligible providers from the central server 18 ordered according to the provider scores of the one or more providers in the list of eligible providers.

In some embodiments, the patient may set an appointment between themselves and the provider by directly selecting a provider from the list of eligible providers.

For purposes of illustrating features of the present embodiments, some simple examples corresponding to the first method as seen in FIG. 4 will now be introduced and referenced throughout the disclosure. Those skilled in the art will recognize that these examples are illustrative and are not limiting and are provided purely for explanatory purposes.

In a first example, Suki is a patient in New York that is looking for a dentist within 5 miles of her apartment. Suki is most comfortable with speaking Japanese. Her employer has provided her with insurance provider X and she needs her dentist to take her insurance.

In a second example, Melissa is a patient in Boston that is also looking for a dentist within 5 miles of her apartment. Melissa wants a native speaking American dentist since she has trouble with accents. Her employer has provided her with insurance provider Y and she needs her dentist to take her insurance.

First, at step 410, one or more patient human factors associated with healthcare are received from the patient. As described above, patient human factors vary widely between patients. These patient human factors may vary according to a patient's region, culture, needs, wants and medical issues. The patient human factors may include one or more of patient expectations, a patient philosophy associated with treatments, patient personal information, patient availability and provider ability, provider philosophy and practice style, prover practice size, culture, provider personal profile and location. These patient human factors may be input as a series of questions that receive typed answers or may be received via voice and then converted to text via a voice to text conversion process.

Continuing with the above examples, patient Suki prefers a dentist that does not use analgesics/anesthetics when performing dental procedures. Suki also prefers natural solutions for pain medications and prefers to avoid pharmaceuticals.

Unlike Suki, patient Melissa prefers to not be awake while the dentist works on her teeth. She wants the whole procedure to be painless and is fine with painkillers and other medications prescribed by the dentist.

While both Suki and Melissa can use conventional means to find dentists that are located in their vicinity, finding out the patient human factors would take multiple phone calls and reviewing online reviews and thus would use considerable network resources.

At step 420, a query to a database is transmitted, via a patient device 12 to the central server 18 having a consolidated provider database. The query includes the patient criteria and the human factors. For example, in the above examples, the patient criteria and the patient human factors for Suki would be transmitted to a database in a query. Likewise, the patient criteria and the patient human factors for Melissa would be transmitted to a database in a second query.

Finally, at step 430, a list of eligible providers based on the query is received by the central server 18 and transmitted to the patient device 12 for viewing.

Turning again to the above examples, the results of the query may be based on the algorithm or algorithm settings that are used for the search. In some embodiments, an administrator of the system may set a weight for each criterion and each factor. Also, a patient may indicate that one criteria or factor is “critical” meaning that if this factor is not met, the provider should not come up in the search results.

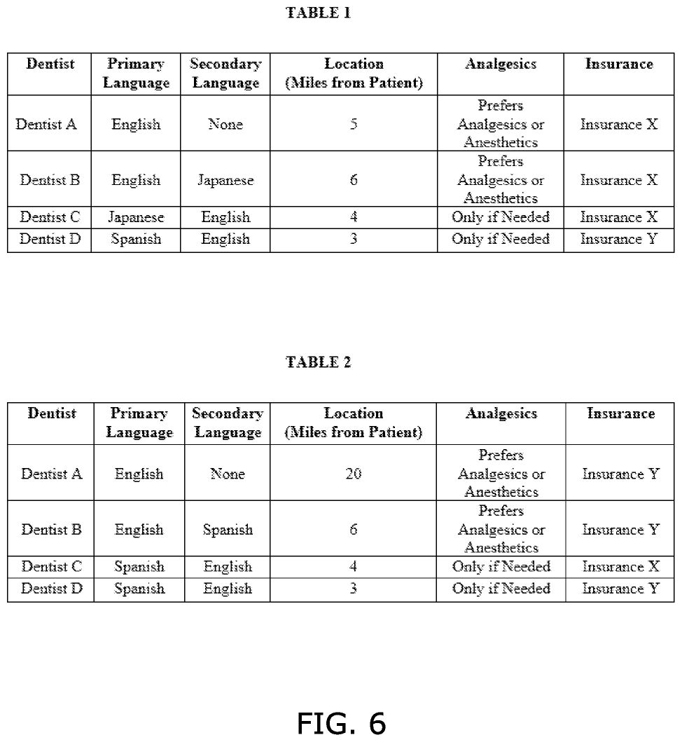

As a simple example, assuming there are four dentists within the search area of Suki, the dentists may have the following criteria and human factor regarding analgesics as seen in Table 1 of FIG. 6.

In Table 1, the administrator has set the weight of each criterion/factor to one. As a result, all matches may have a multiplier of 1 and thus each exact criterion that matches will have a value of 1 and human factors, which may be in the form of key words or phrases, will also have a value of 1.

As stated above, patient Suki prefers a dentist that does not use analgesics when performing dental procedures, is within 5 miles of her apartment, speaks Japanese, and takes insurance provider X. Criteria 3 has been marked as critical for Suki because she needs a dentist that speaks Japanese.

Dentist D and Dentist A did not match on a critical factor so they will not be provided to Suki in the search results. Suki then may see the results from Dentist B and Dentist C. Since Dentist C is less likely to use analgesics (a human factor), Dentist C will be scored higher.

In the second example regarding Melissa, the following dentists are available, as seen in Table 2 of FIG. 6. As stated above, Melissa prefers to not be awake while the dentist works on her teeth so her only critical factor is that the doctor will use analgesics. She wants the whole procedure to be painless and is fine with painkillers and other medications prescribed by the dentist.

In the above example Dentist C and Dentist D did not match on the critical factor so they will not be provided to Melissa in the search results. Melissa then may see the results from Dentist A and Dentist B. Since Dentist B is closer to melissa, Dentist B will be scored higher.

In some embodiments, where one or more patient human factors are not received from the patient, the list of medical providers may be based solely on the distance from the patient, an insurance provider accepted by the medical providers or a combination thereof.

In some embodiments, the present system may also relate to a telemedicine system where once a provider matches with a best fitting patient and the patient matches with a best fitting provider for the patient, an opportunity to engage in a telemedicine session may be presented to the patient. For example, if the provider is currently available after a patient and provider match is made, the patient may be able to immediately initiate a telemedicine session with the provider. However, if a preferred provider is not immediately available, the patient may then be able to make an appointment with the provider for a later date. Thus, for example, if Melissa, from the above-example, is presented with an indication that Dentist B is currently available, Melissa can then start a telemedicine session with Dentist B where Dentist B can help diagnose or discuss Melissa's issues over the phone or computer. The system provides in real-time the optimal provider patient match, and thus the optimal telemedicine session.

This written description uses examples to disclose multiple embodiments, including the preferred embodiments, and also to enable any person skilled in the art to practice the invention, including making and using any devices or systems and performing any incorporated methods. Aspects from the various embodiments described, as well as other known equivalents for each such aspects, can be mixed and matched by one of ordinary skill in the art to construct additional embodiments and techniques in accordance with principles of this application.

The invention also, preferably, relies on feedback to refine potential patient-medical provider matches. Various feedback factors, ideally, should come from patients and providers. One feedback factor is overall satisfaction that may be a number ranging from 1 to 10. Other feedback factors would address what supports or motivates the satisfaction factor, not limited to: 1. What made the patient like/dislike the experience; 2. Whether the provider believed the patient was a good medical fit; 3. What other providers the patient might have preferred. Another feedback factor is whether the patient utilizes again the service or methodology as described herein.

The method or selection process will be directly optimized with the satisfaction score in conjunction with the provider's responses with respect to whether the patient was a good medical fit. If the user provides a low satisfaction score, the additional provided feedback will be incorporated as a noisy training signal to improve sample efficiency. For example, a feedback factor may be added as a keyword and/or criterion, potentially a critical one, for determining initial correspondence among patients and medical providers. The content of the feedback factor may be parsed and, via artificial intelligence, support the introduction of derived keywords and/or criteria.

The like/dislike responses may be leveraged to create a multi task environment to allow for a more robust matching methodology. A multi task environment would allow for greater information per gradient step or sample to be incorporated into the methodology as compared to optimizing a single objective function.

The additional information from the feedback will help with the explainability and refinement of the methodology. To this end, feedback responses may be clustered in semantic spaces and inspected based on the overall performance thereof. For example, a low mean satisfaction score with low variance, in addition to patients not returning to the service, would suggest that critical information is missing, which would necessitate investigation to constructing new features.

Those in the art will appreciate that various adaptations and modifications of the above-described embodiments can be configured without departing from the scope and spirit of the claims. Therefore, it is to be understood that the claims may be practiced other than as specifically described herein.

What is claimed is:

1. A method for reducing network bandwidth related to matching patients with healthcare providers, the method performed by a processor, the method comprising:
collecting, from a patient using a patient device, one or more patient criteria
wherein the patient criteria comprise at least one of a patient insurance carrier, a patient location, a patient maximum distance that they are willing to travel to a provider, and a patient spoken language;
collecting, from the patient using the patient device, one or more patient human factors associated with healthcare;
transmitting, to a central server from the patient device, the one or more patient criteria and the one or more patient human factors;
generating a list of compatible providers, using the central server, by querying a provider database stored on the central server with a query configured to return a list of medical providers from the provider database based on the one or more patient criteria and the one of more patient human factors; and,
transmitting, to the patient device, the list of compatible providers.

2. The method of claim 1, wherein the list of medical providers is generated based on the overlap between the patient criteria and the provider criteria and the overlap between the patient human factors and the provider human factors.

3. The method of claim 1, further comprising:
transmitting, to the patient device from the central server, provider criteria associated with the providers in the list of compatible providers comprising the provider insurance carrier information, the provider location, and the provider spoken language; and,
transmitting, to the patient device from the central server, provider human factors associated with the providers in the list of compatible providers.

4. The method of claim 3, wherein in a case that the one or more patient human factors are not received from the patient, the list of medical providers is based only on a provider's distance from the patient.

5. The method of claim 3, further comprising, prior to transmitting the one or more patient criteria and the one or more patient human factors to the central server:
generating one or more supplementary patient criteria using machine learning techniques stored and executed by a machine learning server communicatively coupled to the central sever based on the one or more patient criteria;
supplementing the one or more patient criteria with the generated one or more supplementary patient criteria; and/or,
generating one or more supplementary patient human factors using machine learning techniques stored and executed by a machine learning server based on the one or more patient human factors; and,
supplementing the one or more patient human factors with the generated one or more supplementary patient human factors.

6. The method of claim 5, further comprising:
generating a matching percentage for each provider from the list of compatible providers calculated as a provider's first matching score divided by the second matching score and then multiplied by 100; and,
transmitting, from the central server to the patient device, the matching percentages associated with the providers in the list of compatible providers;
wherein each provider's second matching score corresponds to the overlap between the patient criteria and the provider criteria and the overlap between the patient human factors and the provider human factors; and,
wherein the second matching score is the greatest second matching score from the set of second matching scores of the providers from the list of compatible providers.

7. The method of claim 6, further comprising, prior to transmitting the one or more patient criteria and the one or more patient human factors to the central server:
collecting, from the patient using the patient device, a plurality of critical data values corresponding to whether the one or more patient criteria and the one or more patient human factors are critical; and,
transmitting, to a central server from the patient device, plurality of critical data values;
wherein the list of compatible providers generated by the central server will only include providers that have provider criteria and provider human factors that match the patient criteria and the patient human factors designated as critical.

8. The method of claim 6, further comprising:
collecting, from the patient using the patient device, a plurality of survey data corresponding to the quality of the generated list of compatible providers; and,
updating the machine learning techniques based on the collected plurality of survey data and/or whether or not the patient uses the system again at a future time.

9. The method of claim 6, wherein the one or more patient criteria and one or more patient human factors are generated by an analytical server from audio data and/or visual data collected from the patient by the patient device wherein the analytical server is communicatively coupled to the central server and/or the patient device.

10. The method of claim 2, further comprising, prior to transmitting the one or more patient criteria and the one or more patient human factors to the central server:
generating supplementary provider criteria using machine learning techniques stored and executed by a machine learning server based on the one or more patient criteria;
supplementing the provider criteria with the supplementary provider criteria; and/or
generating supplementary provider human factors using machine learning techniques stored and executed by the machine learning server based on the one or more patient criteria;
supplementing the provider human factors with the supplementary provider human factors.

11. The method of claim 10, wherein the provider human factors comprise at least one of a provider philosophy, a provider practice style, a provider size, a provider cultural background, and a provider personal profile.

12. The method of claim 11, wherein the patient human factors comprise at least one of a patient expectations, a patient philosophy associated with treatment, and a patient personal information.

13. A system for reducing network bandwidth related to matching patients with healthcare providers, the system comprising:
a patient device configured to collect and transmit a plurality of values of data from a patient;
a central server communicatively coupled to the patient device comprising:
a processor;
a memory configured to receive and store the plurality of value of data from a patient; and,
a non-transitory computer-readable medium comprising processor executable instructions, that when executed by the processor, performs a method, the method comprising:
receiving, from a patient using a patient device, one or more patient criteria wherein the patient criteria comprise at least one of a patient insurance carrier, a patient location, a patient maximum distance that they are willing to travel to a provider, and a patient spoken language;
receiving, from the patient using a patient device, one or more patient human factors associated with healthcare;
transmitting, to a central server from the patient device, the one or more patient criteria and the one or more patient human factors;
generating a list of compatible providers, using the central server, by querying a provider database stored on the central server with a query configured to return a list of medical providers from the provider database based on the one or more patient criteria and the one of more patient human factors; and,
transmitting, to the patient device, the list of compatible providers.

14. The system of claim 13, wherein the method further comprises:
prior to transmitting the one or more patient criteria and the one or more patient human factors to the central server:
generating one or more supplementary patient criteria using machine learning techniques stored and executed by a machine learning server based on the one or more patient criteria;
supplementing the one or more patient criteria with the generated one or more supplementary patient criteria; and/or,
generating one or more supplementary patient human factors using machine learning techniques stored and executed by a machine learning server based on the one or more patient human factors;
supplementing the one or more patient human factors with the generated one or more supplementary patient human factors.

15. The system of claim 14, wherein the method further comprises:
receiving, from the central server by the patient device, provider criteria comprising the provider insurance carrier information, the provider location, and
the provider spoken language; and,
receiving, from the central server by the patient device, provider human factors associated with healthcare wherein the list of medical providers is based on matching the patient criteria with the provider criteria and the patient human factors with the provider human factors.

16. The system of claim 14, further comprising:
generating a matching percentage for each provider from the list of compatible providers calculated as a provider's first matching score divided by the second matching score and then multiplied by 100; and,
transmitting, from the central server to the patient device, the matching percentages associated with the providers in the list of compatible providers;
wherein each provider's second matching score corresponds to the overlap between the patient criteria and the provider criteria and the overlap between the patient human factors and the provider human factors; and,
wherein the second matching score is the greatest second matching score from the set of second matching scores of the providers from the list of compatible providers.

17. The system of claim 14, further comprising:
collecting, from the patient using the patient device, a plurality of critical data values corresponding to whether the one or more patient criteria and the one or more patient human factors are critical; and,
transmitting, to a central server from the patient device, plurality of critical data values;
wherein the list of compatible providers generated by the central server will only include providers that have provider criteria and provider human factors that match the patient criteria and the patient human factors designated as critical.

18. The system of claim 14, further comprising an analytical server communicatively coupled to the central server and/or the patient device, wherein the one or more patient criteria and one or more patient human factors are generated by an analytical server from audio data and/or visual data collected from the patient by the patient device.

19. The system of claim 14, wherein the provider human factors comprise at least one of patient expectations, a patient philosophy associated with treatments, and a patient personal information, wherein the provider human factors comprise at least one of a provider philosophy, a provider practice style, a provider size, a provider cultural background, and/or a provider personal profile, and wherein in a case that that the one or more patient human factors are not received from the patient, the list of medical providers is based on the distance of the providers from the patient.

20. A non-transitory computer-readable medium, which stores at least the instructions that are executed by a processor to induce the system to:
access data corresponding to patient criteria;
identify at least one provider having a provider criterion that corresponds with a patient criterion, defining an eligible provider set;
for each provider in the eligible provider set:
for each provider criterion that corresponds with a patient criterion:
assessing a correspondence of a value of the patient criterion relative to the value of the corresponding provider criterion and defining a criterion score;
identifying a maximum value for the patient criterion and defining a maximum score;
dividing a sum of the criteria scores by a sum of the maximum scores, defining a provider score;
displaying data pertaining to providers of the eligible provider set ordered by the provider score;
displaying data pertaining to providers of the eligible provider set ordered by provider score; and,
in response to a selection respecting a medical provider from a display, setting an appointment for the patient and provider based on the selection.
